(19) United States
(12) Patent Application Publication
(10) Pub. No.: US 2005/0214280 A1
(43) Pub. Date: Sep. 29, 2005

(54) Method for inhibiting tumor formation and growth

(75) Inventors: Xuri Li, Stockholm (SE); Ulf Eriksson, Stockholm (SE)

Correspondence Address: CROWELL & MORING LLP;INTELLECTUAL PROPERTY GROUP, P.O. BOX 14300, WASHINGTON, DC, 20044-4300, US

(73) Assignee: LUDWIG INSTITUTE FOR CANCER RESEARCH, New York, NY (US)

(21) Appl. No.: 11/068,888

(22) Filed: Mar. 2, 2005

Related U.S. Application Data
(60) Provisional application No. 60/548,864, filed on Mar. 2, 2004.

(51) Int. Cl. A61K 39/395

(52) U.S. Cl. 424/141.1; 424/145.1

(57) ABSTRACT

Method for inhibiting tumor cell formation or tumor cell growth, the method comprising administering to a patient in need thereof an antagonist to VEGF-B. Preferably, the antagonist is an anti-VEGF-B antibody, an antisense molecule, an RNAi molecule, a molecule for forming a triplex nucleic acid molecule with a VEGF-B encoding polynucleotide. Also disclosed are pharmaceutical compositions comprising the same.

### CROSS-REFERENCES TO RELATED APPLICATIONS

This application claims priority of U.S. Provisional Application Ser. No. 60/548,864, filed Mar. 2, 2004, the content of which is incorporated herein by reference in its entirety.

### BACKGROUND OF THE INVENTION

Angiogenesis is a process of new blood vessel formation by growth and branching of pre-existing vessels. It is important in late embryogenesis and is responsible for blood vessel growth in the adult. Angiogenesis is a physiologically complex process involving proliferation of endothelial cells, degradation of extracellular matrix, branching of vessels and subsequent cell adhesion events. In the adult, angiogenesis is tightly controlled and limited under normal circumstances to the female reproductive system. However angiogenesis can be switched on in response to tissue damage. The molecular mechanisms underlying the complex angiogenic processes are far from being understood.

Angiogenesis is also involved in a number of pathologic conditions, where it plays a role or is involved directly in the diseases. For example, solid tumors are able to induce angiogenesis in surrounding tissue, thus sustaining tumor growth and facilitating the formation of metastases (Folkman, J., Nature Med. 1:27-31, (1995)). Because of the crucial role of angiogenesis in so many physiological and pathological processes, factors involved in the control of angiogenesis have been intensively investigated. For a detailed discussion of angiogenesis, the growth factors known to be involved, their respective receptors, and tissue-specific and development-specific expression and regulation of these factors and their receptors, see U.S. Pat. No. 6,670,125, incorporated herein by reference.

The Vascular Endothelial Growth Factors (VEGFs) and their corresponding receptors are primarily responsible for stimulation of endothelial cell growth and differentiation, and for certain functions of the differentiated cells. These factors appear to act primarily via endothelial receptor tyrosine kinases (RTKs). Eight different proteins have been identified in the PDGF/VEGF family, namely two platelet derived growth factors (PDGFs A and B), VEGF and five closely related members: VEGF-B; VEGF-C or VEGF2; VEGF-D; the placenta growth factor (PlGF); and VEGF3.

Vascular endothelial growth factor B (VEGF-B) is a non-glycosylated, highly basic growth factor. VEGF-B has similar angiogenic and other properties to those of VEGF, but is distributed and expressed in tissues differently from VEGF. In particular, VEGF-B is very strongly expressed in heart, and only weakly in lung, whereas the reverse is the case for VEGF (Olofsson, B. et al, Proc. Natl. Acad. Sci. USA 93:2576-2581 (1996).

It has been shown that members of the VEGF/PDGF family produce variant transcripts. VEGF has been shown to display different transcripts because of alternative splicing. For example, the human VEGF gene has five different mRNA species (Neufeld et al, FASEB J. 13:9-22 (1999)), resulting in proteins differing in their molecular mass and biological properties (Carmeliet, P., Nat. Med. 6:389-395 (2000)), and the placenta growth factor (PlGF) has three different isoforms, which are expressed in a tissue and development specific way (Maglione et al, Oncogene 8:925-31 (1993); Cao et al., Biochem. Biophys. Res. Commun. 235:493-8 (1997)).

Presently, two isoforms of VEGF-B, generated by alternative splicing of mRNA, have been recognized: they are a cell associated form of 167 amino acid residues (VEGF-B₁₆₇) and a secreted form of 186 amino acid residues (VEGF-B₁₈₆). The isolation of the human and mouse VEGF-B isoforms are described in detail in PCT/US96/02957, in U.S. Pat. Nos. 5,840,693 and 5,607,918, and in Olofsson et al, Proc. Natl. Acad. Sci. USA 93:2576-2581 (1996). The present inventors have previously discovered that there are two splicing variants, VEGF-B₁₆₇ and VEGF-B₁₈₆, and that while VEGF-B₁₆₇ is the major isoform expressed almost universally both in adult and embryonic tissues, VEGF-B₁₈₆ is expressed at a lower level in a tissue-specific manner, and such tissue-specific expression appears to be development independent (i.e. the expression pattern in the various tissues appears to not change as the animal undergoes developmental changes). See U.S. Pat. No. 6,670,125. The present inventors have also discovered that VEGF-B₁₈₆ is up-regulated in various tumor cells lines, such as fibrosarcoma, melanoma, Lewis lung carcinoma, glioma, and pheochromocytoma. Further, such up-regulation occurs in benign tumor cells as well as in malignant cells.

Five endothelial cell-specific receptor tyrosine kinases have been identified, belonging to two distinct subclasses: three vascular endothelial cell growth factor receptors, VEGFR-1 (Flt-1), VEGFR-2 (KDR/Flk-1), VEGFR-3 (Flt4), and the two receptors of the Tie family, Tie and Tie-2 (Tek). These receptors differ in their specificity and affinity. All of these have the intrinsic tyrosine kinase activity which is necessary for signal transduction.

The only receptor tyrosine kinases known to bind VEGFs are VEGFR-1, VEGFR-2 and VEGFR-3. VEGFR-1, VEGFR-2 and VEGFR-3 are expressed differently by endothelial cells. Generally, both VEGFR-1 and VEGFR-2 are expressed in blood vessel endothelia (Oelrichs et al, Oncogene 8:11-18 (1992); Kaipainen et al, J. Exp. Med. 178:2077-2088 (1993); Dumont et al, Dev. Dyn. 203:80-92 (1995); Fong et al, Dev. Dyn. 207:1-10 (1996)) and VEGFR-3 is mostly expressed in the lymphatic endothelium of adult tissues (Kaipainen et al, Proc. Natl. Acad. Sci. USA 9:3566-3570 (1995)). VEGFR-3 is also expressed in the blood vasculature surrounding tumors.

VEGF-B binds to VEGFR-1 with high affinity, but not to VEGFR-2 or -3 (Olofsson et al, Proc. Natl. Acad. Sci. USA, 95:11709-11714 (1998)). Although VEGFR-1 is mainly expressed in endothelial cells during development, it can also be found in hematopoetic precursor cells during early stages of embryogenesis (Fong et al, Nature 376:66-70 (1995)). In adults, monocytes and macrophages also express this receptor (Barleon et al, Blood 87:3336-3343 (1995)). In embryos, VEGFR-1 is expressed by most, if not all, vessels (Breier et al, Dev. Dyn. 204:228-239 (1995); Fong et al, Dev. Dyn. 207:1-10 (1996)).

An important early finding involves the connection between angiogenesis and tumor development. Both tumor growth and metastasis are angiogenesis-dependent processes (Folkman, J. and Shing, Y., J. Biol. Chem. 267: 10931-10934 (1992)). For example, when tumor cells are introduced into an animal, the expression pattern of VEGF mRNA reveals expression at the highest level in cells at the periphery of necrotic, tumor growth areas. Numerous blood vessels were identified within these areas. The expression of VEGF in these areas suggests that hypoxemia, a state of deficient oxygenation, triggers expression and release of VEGF in the necrotic tumor.

The expression of VEGF-B also has been directly correlated with tumor growth (see U.S. Pat. No. 5,840,693). VEGF-B expression is especially up regulated in tumor-associated macrophages and also in ovarian epithelial tumors (Sowter et al, Lab Invest. 77:607-14, (1997)). VEGF-B mRNA can be detected in most tumor cell lines investigated, including adenocarcinoma, breast carcinoma, lymphoma, squamous cell carcinoma, melanoma, fibrosarcoma and Schwannoma (Salven et al, Am. J. Pathol. 153:103-8 (1998)).

Published data suggest that VEGFR-1 plays a direct role in tumor angiogenesis. Although these known data suggest that inhibition of VEGFR-1 may be effective in inhibiting tumor growth, concerns exist over the effectiveness and possible deleterious side effects of this approach because VEGFR-1 is known to bind and be activated by many other growth factors, and have many important biological functions which are not related to tumor angiogenesis.

VEGF-B is known to be a weak angiogenic growth factor that binds and activates VEGFR-1. RT-PCR assays have demonstrated the presence of VEGF-B mRNA in melanoma, normal skin, and muscle. Prior to this instant invention, there has been no evidence suggesting that inhibition of VEGF-B directly would inhibit tumor angiogenesis, tumor formation or tumor stromal growth.

### BRIEF DESCRIPTION OF THE DRAWINGS

FIG. 1 shows an immunoblot analysis of VEGF-B species released from transformed normal (VEGF-B+/+) and VEGF-B deficient mouse embryonic fibroblasts (MEFs). Aliquots of conditioned media from cell cultures were subjected to TCA-precipitation, SDS-PAGE under reducing conditions, and immunoblotting using a rabbit antiserum to mouse VEGF-B. The transformed normal MEFs secreted mainly VEGF-B 186 isoform.

FIG. 2 shows the growth of transformed normal (VEGF-B+/+) and VEGF-B deficient (VEGF-B−/−) MEFs on normal and VEGF-B deficient animals. No growth of the tumor cells occurred in the absence of host and tumor-derived VEGF-B suggesting a crucial role of VEGF-B to initiate and maintain tumor growth.

FIG. 3 shows that tumor growth is normal in VEGF-B-deficient mice and upregulation of VEGF-B stimulates tumor growth. (a) Murine T241 fibrosarcoma cells (which naturally express both the 167 and 186 isoforms of VEGF-B) were transplanted (1×10⁶ cells) into both wild-type (+/+) and VEGF-B KO mice (−/−) and the growth of the tumor was followed. There was no difference is tumor growth showing that in this case the background expression of host VEGF-B does not affect tumor growth; (b) and (c) An antibody to PECAM was used to stain tissue sections from the transplanted tumors which showed that there was no difference in vessel density in cells transplanted into either WT (for VEGF-B) or VEGF-B KO mice; (d) murine VEGF-B167 was cloned into the pcDNA3.1 expression vector which was transfected into T231 cells using lipofectin. Clones were selected using neomycin (400 micrograms/ml). The figure shows a western blot using an antibody to VEGF-B (which recognizes both 186 and 167 isoforms (see Aase et al., 1999, Developmental Dynamics 215:12-25). Lanes: mock-transfected (vector alone); and two different transfectant clones (#3 and #8) which have different levels of expression of VEGF-B₁₆₇. #8 has intermediate levels of expression; #3 has high levels of expression. VEGF-B₁₈₆ levels of expression are low; and (e) Transfected cells in (d) were put into WT VEGF-B mice and growth of the tumor assessed: Clone #3 showed accelerated growth of tumor versus mock transfected cells showing that tumor growth is dependent on VEGF-B₁₆₇ expression. The same experiment was done with the 186 isoform and results showed that tumor growth was even more accelerated.

FIG. 4 shows that monoclonal antibody MAB3372 is specific to human, but not murine VEGF-B proteins.

FIG. 5 shows that MAB3372 inhibits VEGF-B-induced viability of Ba/F3 cells expressing a chimeric VEGFR-1/EpoR.

FIG. 6 shows a comparison of the tumorigenicity of mock-transfected T241 and T241 cells over-expressing the two isoforms of VEGF-B.

FIG. 7 shows the growth rate of tumors expressing each VEGF-B isoform.

FIG. 8 shows a statistical analysis of the data in FIG. 7.

FIG. 9 shows the number of anti-F4/80 antibody positive macrophages within T241 and T241-VEGF-B induced tumor tissues.

FIG. 10A shows the tumorigenicity of wild-type and VEGF-B deficient transformed murine embryonic fibroblasts; and FIG. 10B compares the number of anti-F4/80 antibody positive macrophages within wild-type and VEGF-B deficient fibrosarcoma cells induced tumor tissues.

### DESCRIPTION OF THE INVENTION

The present inventors employed a genetic approach, and directly demonstrated a role of VEGF-B in tumor growth and angiogenesis. Specifically, MEFs from normal and VEGF-B deficient animals were transformed using established protocols and the growth of the transformed MEFs was analyzed in normal and VEGF-B deficient animals. The data demonstrate that VEGF-B deficient transformed MEFs failed to grow in VEGF-B deficient mice, suggesting an essential role of VEGF-B in tumor establishment and growth. Accordingly, this invention provides antagonizing compositions capable of inhibiting the expression or activity of VEGF-B, as well as methods for treating cancers or tumors associated with increased VEGF-B quantity or activity.

The data further demonstrate that inhibition of VEGF-B expression or function reduces recruitment of macrophage to the tumor site. Accordingly, methods and compositions of this invention capable of inhibiting the expression or activity of VEGF-B can also be used for inhibiting macrophage recruitment or invasion to a particular tissue, and for treating inflammatory reactions that occur in, for example, rheumatoid arthritis, inflammatory bowel disease, Crohn's disease, multiple sclerosis and psoriasis. The methods and compositions of the present invention can also be employed in the treatment of diseases wherein macrophages are an essential element of the disease process. Animal models for many pathological conditions associated with macrophage activity have been described in the art, and in each of these models, the disease state can be attenuated by inhibiting an interleukin activity, which presumably also inactivated macrophage activities. For example, in mice, macrophage recruitment to sites of both chronic and acute inflammation is reported by Jutila et al., J. Leukocyte Biol. 54:30-39 (1993). In rats, Adams et al., Transplantation 53:1115-1119(1992) and Transplantation 56:794-799 (1993), describe a model for graft arteriosclerosis following heterotropic abdominal cardiac allograft transplantation. Rosenfeld et al., Arteriosclerosis 7:9-23 (1987) and Arteriosclerosis 7:24-34 (1987), describe induced atherosclerosis in rabbits fed a cholesterol supplemented diet. Hanenberg et al., Diabetologia 32:126-134 (1989), report the spontaneous development of insulin-dependent diabetes in BB rats. Yamada et al., Gastroenterology 104:759-771 (1993), describe an induced inflammatory bowel disease, chronic granulomatous colitis, in rats following injections of streptococcal peptidoglycan-polysaccharide polymers. Cromartie, et al., J. Exp. Med. 146:1585-1602 (1977), and Schwab et al., Infection and Immunity 59:4436-4442 (1991) report that injection of streptococcal cell wall protein into rats results in an arthritic condition characterized by inflammation of peripheral joints and subsequent joint destruction. Finally, Huitinga et al., Eur. J. Immunol 23:709-715 (1993) describe experimental allergic encephalomyclitis, a model for multiple sclerosis, in Lewis rats.

The term VEGF-B includes any and all isoforms of VEGF-B, including a polypeptide molecule having a VEGF-B function of any origin (e.g. human, murine or any other mammal), a functional fragment or derivative thereof, the nucleic acid molecule encoding the same, and any and all functional equivalent known or to be discovered in the future.

In another aspect, the invention provides a method of inhibiting cancer formation or growth in a mammal, by administering thereto a dosage of a VEGF-B antagonist, or an anti-VEGF-B agent, such as anti-VEGF-B antibodies, a therapeutic nucleic acid (antisense, ribozyme, small interfering RNA molecules (RNAi or siRNA), dsRNA, and triple helix molecules) molecule wherein the administered nucleic acid inhibits the expression of VEGF-B and small molecules.

The term “VEGF-B antagonist,” “anti-VEGF-B agent,” or “VEGF-B antagonizing agent” herein means any composition that inhibits or blocks VEGF-B expression, production, mRNA splicing, dimerization, or secretion, or any composition that inhibits or blocks the biological activity of VEGF-B, such as its binding to VEGFR-1.

VEGF-B antagonizing agents include any reagent or molecule inhibiting VEGF-B expression or production including but not limited to: (1) antisense VEGF-B DNA or RNA molecules that inhibit VEGF-B expression by inhibiting VEGF-B translation; (2) reagents (hormones, growth factors, small molecules) that inhibit VEGF-B mRNA and/or protein expression at the transcriptional, translational or post-translational levels; and (3) factors, or reagents that inhibit VEGF-B secretion.

VEGF-B antagonizing agents also include any reagent or molecule that will inhibit VEGF-B action or biological activity such as (1) neutralizing antibodies to VEGF-B that bind the protein and prevent it from exerting its biological activity or that bind VEGF-B monomers and prevent them from dimerizing or that allow dimerization but prevent receptor binding; (2) antibodies to the VEGF-B receptor that prevent VEGF-B from binding to its receptor and from exerting its biological activity or that prevent receptor dimerization; (3) competitive inhibitors of VEGF-B binding to its receptors (e.g., proteins, ribozymes, aptamers, small molecules); and (4) inhibitors of VEGF-B signaling pathways (e.g., proteins, ribozymes, aptamers, small molecules).

In one embodiment, this invention provides neutralizing antibodies to inhibit VEGF-B biological action. In another embodiment of the invention, the VEGF-B antagonizing agents are antisense oligonucleotides to VEGF-B. The antisense oligonucleotides preferably inhibit VEGF-B expression by inhibiting translation of the VEGF-B protein. In a further embodiment, the antagonizing agent is RNAi (RNA interference nucleic acids) or siRNA (small interfering RNA molecules). RNAi are double-stranded RNA molecules, typically 21 n.t. in length, that are homologous to the target gene (e.g., VEGF-B) and interfere with the target gene's activity.

Alternatively, such a composition may comprise reagents or factors that inhibit VEGF-B expression by regulating VEGF-B gene transcriptional activity. Such a composition may comprise reagents, factors or hormones that inhibit VEGF-B post-translational modification and its secretion. Such a composition may comprise reagents that act as VEGF-B antagonists that block VEGF-B activity by competing with VEGF-B for binding to VEGF-B cell surface receptors. Alternatively, such a composition may comprise factors or reagents that inhibit the signaling pathway transduced by VEGF-B once bound to its receptors on cells.

The composition may also comprise reagents that block VEGF-B action such as an antibody to the VEGF-B receptor, e.g. VEGFR-1, that blocks its activity.

The antagonisits of the invention (neutralizing and others) are preferably used as a treatment for cancer formation or growth. A preferred embodiment is a neutralizing antibody. By the term “neutralizing” it shall be understood that the antibody has the ability to inhibit or block the normal biological activity of VEGF-B.

An anti-VEGF-B antibody suitable for the present invention may be a polyclonal antibody. Preferably, the antibody is a monoclonal antibody. The antibody may also be isoform-specific, such as those disclosed in U.S. Pat. No. 6,670,125.

The monoclonal antibody or binding fragment thereof of the invention may be Fab fragments, F(ab)₂ fragments, Fab′ fragments, F(ab′)₂ fragments, Fd fragments, Fd′ fragments or Fv fragments. It may also be an anti-idiotypic antibody. The antibody may be labeled with a detectable moiety, such as a fluorophore, a chromophore, a radionuclide, a chemiluminescent agent, a bioluminescent agent and an enzyme. Domain antibodies (dAbs) (for review, see Holt et al., 2003, Trends in Biotechnology 21:484-490) are also suitable for the methods of the present invention.

Various methods of producing antibodies with a known antigen are well-known to those ordinarily skilled in the art (see for example, Harlow and Lane, 1988, Antibodies: A Laboratory Manual. Cold Spring Harbor Laboratory Press, Cold Spring Harbor, N.Y.; see also WO 01/25437). In particular, suitable antibodies may be produced by chemical synthesis, by intracellular immunization (i.e., intrabody technology), or preferably, by recombinant expression techniques. Methods of producing antibodies may further include the hybridoma technology well-known in the art.

In accordance with the present invention, the antibodies or binding fragments thereof may be characterized as those which are capable of specific binding to a VEGF-B or an antigenic fragment thereof, preferably an epitope that is recognized by an antibody when the antibody is administered in vivo. Antibodies can be elicited in an animal host by immunization with a VEGF-B-derived immunogenic components, or can be formed by in vitro immunization (sensitization) of immune cells. The antibodies can also be produced in recombinant systems in which the appropriate cell lines are transformed, transfected, infected or transduced with appropriate antibody-encoding DNA. Alternatively, the antibodies can be constructed by biochemical reconstitution of purified heavy and light chains.

The antibodies may be human, or from animals other than humans, preferably mammals, such as rat, mouse, guinea pig, rabbit, goat, sheep, and pig. Preferred are mouse monoclonal antibodies and antigen-binding fragments or portions thereof. In addition to fully human antibodies, humanized, chimeric and hybrid antibodies are also embraced by the present invention. Techniques for the production of chimeric antibodies are described in e.g. Morrison et al., 1984, Proc. Natl. Acad. Sci. USA, 81:6851-6855; Neuberger et al., 1984, Nature, 312:604-608; and Takeda et al., 1985, Nature, 314:452-454.

Further, single chain antibodies are also suitable for the present invention (e.g., U.S. Pat. Nos. 5,476,786 and 5,132,405 to Huston; Huston et al., 1988, Proc. Natl. Acad. Sci. USA, 85:5879-5883; U.S. Pat. No. 4,946,778 to Ladner et al.; Bird, 1988, Science, 242:423-426 and Ward et al., 1989, Nature, 334:544-546). Single chain antibodies are formed by linking the heavy and light immunoglobulin chain fragments of the Fv region via an amino acid bridge, resulting in a single chain polypeptide. Univalent antibodies are also embraced by the present invention. Such fragments can be obtained by screening using phage display libraries, e.g. libraries commercially available from Dyax Corp (Cambridge, Mass.), Cambridge Antibody Technologies (Cambridge, UK) or may be generated as described in U.S. Pat. Nos. 6,319,690 or 6,300,064.

Anti-VEGF-B antibodies may be administered together with, before or after chemotherapeutic agents including but not limited to 5-fluorouracil or cisplatin. The antibodies can also be administered in combination, before or after administration of antagonists to VEGF-R1. Furthermore, these antibodies and antagonists may be used with, before or after antibodies or antagonists to other members of the PDGF/VEGF family, such as but not limited to VEGF-A, VEGF-C, VEGF-D, VEGF-E, PDGF-A, PDGF-B, PDGF-C and PDGF-D.

Many routes of delivery are known to the skilled artisan for delivery of anti-VEGF-B antibodies. For example, direct injection may be suitable for delivering the antibody to the site of interest. It is also possible to utilize liposomes with antibodies in their membranes to specifically deliver the liposome to the area of the tumor where VEGF-B expression or function is to be inhibited. These liposomes can be produced such that they contain, in addition to monoclonal antibody, other therapeutic agents, such as those described above, which would then be released at the tumor site (e.g., Wolff et al., 1984, Biochem. et Biophys. Acta, 802:259).

This invention also provides VEGF-B antisense nucleic acid molecules and compositions comprising such antisense molecules. The constitutive expression of antisense RNA in cells has been known to inhibit the gene expression, possibly via the blockage of translation or prevention of splicing. Interference with splicing allows the use of intron sequences which should be less conserved and therefore result in greater specificity, inhibiting expression of a gene product of one species but not its homologue in another species.

The term antisense component corresponds to an RNA sequence as well as a DNA sequence coding therefor, which is sufficiently complementary to a particular mRNA molecule, for which the antisense RNA is specific, to cause molecular hybridization between the antisense RNA and the mRNA such that translation of the mRNA is inhibited. Such hybridization can occur under in vivo conditions. This antisense molecule must have sufficient complementarity, about 18-30 nucleotides in length, to the VEGF-B gene so that the antisense RNA can hybridize to the VEGF-B gene (or mRNA) and inhibit VEGF-B gene expression regardless of whether the action is at the level of splicing, transcription, or translation. The antisense components of the present invention may be hybridizable to any of several portions of the target VEGF-B cDNA, including the coding sequence, 3′ or 5′ untranslated regions, or other intronic sequences, or to VEGF-B mRNA.

Because the length of the antisense molecule is short, it can be made isoform specific. In other words, it is possible to devise an antisense molecule that specifically inhibits VEGF-B₁₈₆, but not VEGF-B₁₆₇, or vice versa.

Antisense RNA is delivered to a cell by transformation or transfection via a vector, including retroviral, adenoviral, or adeno-associated viral vectors, and plasmids, into which has been placed DNA encoding the antisense RNA with the appropriate regulatory sequences including a promoter to result in expression of the antisense RNA in a host cell. In one embodiment, stable transfection and constitutive expression of vectors containing VEGF-B cDNA fragments in the antisense orientation are achieved, or such expression may be under the control of tissue or development-specific promoters. Delivery can also be achieved by liposomes.

For in vivo therapy, a preferred method is direct delivery of antisense oligonucleotides, instead of stable transfection of an antisense cDNA fragment constructed into an expression vector. Antisense oligonucleotides having a size of 15-30 bases in length and with sequences hybridizable to any of several portions of the target VEGF-B cDNA, including the coding sequence, 3′ or 5′ untranslated regions, or other intronic sequences, or to VEGF-B mRNA, are preferred. Sequences for the antisense oligonucleotides to VEGF-B are preferably selected as being the ones that have the most potent antisense effects. Factors that govern a target site for the antisense oligonucleotide sequence include the length of the oligonucleotide, binding affinity, and accessibility of the target sequence. Sequences may be screened in vitro for potency of their antisense activity by measuring inhibition of VEGF-B protein translation and VEGF-B related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides.

Preferred VEGF-B antisense oligonucleotides are those oligonucleotides which are stable, have a high resilience to nucleases (enzymes that could potentially degrade oligonucleotides), possess suitable pharmacokinetics to allow them to traffic to target tissue site at non-toxic doses, and have the ability to cross through plasma membranes.

Phosphorothioate antisense oligonucleotides may be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNase H. Its basic structure is also amenable to modifications that may allow its optimization as an antisense component. With respect to modifications of the heterocycle, certain heterocycle modifications have proven to augment antisense effects without interfering with RNase H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

The delivery route will be the one that provides the best antisense effect as measured according to the criteria described above. In vitro cell culture assays and in vivo tumor growth assays using antisense oligonucleotides have shown that delivery mediated by cationic liposomes, by retroviral vectors and direct delivery are efficient. Another possible delivery mode is targeting using antibody to cell surface markers for the tumor cells. Antibody to VEGF-B or to its receptor may serve this purpose.

Alternatively, nucleic acid sequences which inhibit or interfere with gene expression (e.g., RNAi, ribozymes, aptamers) can be used to inhibit or interfere with the activity of RNA or DNA encoding VEGF-B.

RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′, 5′ oligoadenylate synthetase (2′,5′-AS), which synthesizes a molecule that activates RNase L, a nonspecific enzyme that targets all mRNAs. The nonspecific pathway may represents a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al. (1975) J. Biol. Chem. 250: 409-17; Manche et al. (1992) Mol. Cell Biol. 12: 5239-48; Minks et al. (1979) J. Biol. Chem. 254: 10180-3; and Elbashir et al. (2001) Nature 411: 494-8). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base. pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a VEGF-B nucleic acid.

Similar to the antisense molecules discussed above, RNAi suitable for the present invention may also be made isoform-specific.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions of the invention (see below).

The dsRNA oligonucleotides may be introduced into the cell by transfection with an heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141: 863-74). The effectiveness of the RNAi may be assessed by any of a number of assays following introduction of the dsRNAs. These include Western blot analysis using antibodies which recognize the VEGF-B gene product following sufficient time for turnover of the endogenous pool after new protein synthesis is repressed, reverse transcriptase polymerase chain reaction and Northern blot analysis to determine the level of existing VEGF-B target mRNA.

Further compositions, methods and applications of RNAi technology are provided in U.S. Pat. Nos. 6,278,039, 5,723,750 and 5,244,805, which are incorporated herein by reference.

Ribozymes are enzymatic RNA molecules capable of catalyzing specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a VEGF-B mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave VEGF-B mRNA transcripts can also be used to prevent translation of subject VEGF-B mRNAs.

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach, 1988, Nature 334:585-591; and WO89/05852, the contents of which are incorporated herein by reference. Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J. Virol. 73: 1868-77; Kuwabara et al. (1999) Proc. Natl. Acad. Sci. USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the VEGF-B mRNA would allow the selective targeting of one or the other VEGF-B isoforms.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a VEGF-B mRNA. In addition, ribozymes possess highly specific endoribonuclease activity, which autocatalytically cleaves the target sense mRNA.

The ribozymes of the present invention also include RNA endoribonucleases (“Cech-type ribozymes”) such as the one which occurs naturally in Tetrahymena thermophila (known as the IVS, or L-19 IVS RNA) and which has been extensively described in Zaug et al. (1984) Science 224:574-578; Zaug et al. (1986) Science 231:470-475; Zaug et al. (1986) Nature 324:429-433; WO 88/04300; and Been, et al. (1986) Cell 47:207-216). The Cech-type ribozymes have an eight base pair active site which hybridizes to a target RNA sequence where cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a VEGF-B nucleic acid.

In a long target RNA chain, significant numbers of target sites are not accessible to the ribozyme because they are hidden within secondary or tertiary structures (Birikh et al. (1997) Eur. J. Biochem. 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol. 183: 281-306). Other approaches utilize a systematic approach to predicting secondary structure which involves assessing a huge number of candidate hybridizing oligonucleotides molecules (see Milner et al. (1997) Nat. Biotechnol. 15: 537-41; and Patzel and Sczakiel (1998) Nat. Biotechnol. 16: 64-8). Additionally, U.S. Pat. No. 6,251,588, the contents of which are herein incorporated by reference, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the invention.

Alternatively, VEGF-B gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the VEGF-B sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of VEGF-B gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo are similar methods of delivery RNA ribozyme, as outlined in detail above. Additionally, one of skill in the art will recognize that, like antisense oligonucleotide, DNA enzymes can be optionally modified to improve stability and improve resistance to degradation.

The dosage ranges for the administration of the antagonists of the invention are those large enough to produce the desired effect. The dosage should not be so large as to cause adverse side effects, such as unwanted cross-reactions, anaphylactic reactions, and the like. Generally, the dosage will vary with the age, condition, sex and extent of disease of the patient and can be determined by one of skill in the art. The dosage can be adjusted by the individual physician in the event of any complication.

The antagonists of the invention can be administered parenterally by injection or by gradual perfusion over time. The antagonists can be administered intravenously, intraperitoneally, intramuscularly, subcutaneously, intracavity, or transdermally.

Another embodiment of the present invention relates to pharmaceutical compositions comprising one or more antagonists, according to the invention, together with a physiologically- and/or pharmaceutically-acceptable carrier, excipient, or diluent. Physiologically acceptable carriers, excipients, or stabilizers are known to those skilled in the art (see Remington's Pharmaceutical Sciences, 17th edition, (Ed.) A. Osol, Mack Publishing Company, Easton, Pa., 1985). Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

### EXAMPLES

### Example 1

### In Vitro Growth and Transformation of MEFs Lead to Induction of VEGF-B Expression, and VEGF-B is Required to Initiate and Support Tumor Growth

1. Cell Lines and Cell Culture

Mouse embryonic fibroblast cells (MEFs) from VEGF-B deficient (1) and wild type embryos (E12) were isolated as described previously (2). The cells were grown in DMEM medium (Life Technologies, Inc.) with 10% fetal bovine serum (Life Technologies, Inc.), glutamine, and penicillin/streptomycin. The MEFs were immortalized after co-transfection with SV40 T antigen (3), and a hygromycin resistance gene, and selected in hygromycin-containing medium (120 μg/ml) for one week. The resistant MEFs were then infected with H-ras61L retrovirus, and selected the next day in puromycin-containing medium (4 μg/ml) for one week (The plasmids and retrovirus were kindly provided by Dr. P. Carmeliet of the Flanders Interuniversity Institute for Biotechnology).

All transformed cells, including the MEF cells from VEGF-B −/− animals, are immortal. They continued to grow for many passages in vitro, and more importantly as foci in soft agar, a classical criteria for transformed cells.

2. Immunoblot Analysis of VEGF-B Expression

Cultures of the transformed MEFs were treated overnight with 100 μg/ml of heparin and 1 ml fractions of serum-free conditioned media were subjected to TCA-precipitation and analyzed by SDS-PAGE under reducing conditions. Immunoblotting was done using a rabbit antiserum to mouse VEGF-B (4).

3. Tumorigenicity Assays

Immortalized MEF cells (5×10⁶) in 150 μl PBS were injected subcutaneously on the back into VEGF-B deficient and wild type mice. Tumors were harvested at 21 days post-injection and tumors dissected and weighed.

It is noted that other tumors, such as T241 fibrosarcoma, B16 melanoma, and LLC (Lewis Lung Carcinoma) were able to grow in VEGF-B −/− mice. In other words, VEGF-B −/− animals do not have any general defect in their ability to support tumor growth.

4. Results

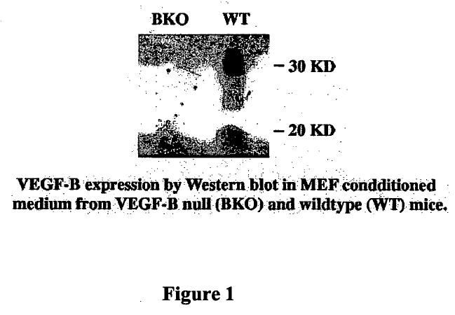

Immunoblot analysis revealed that the transformed MEFs from normal mice expressed a major 32 kDa species of VEGF-B (FIG. 1). This species corresponds to VEGF-B₁₈₆ (5). In addition, a minor 22 kDa species, corresponding to VEGF-B₁₆₇ was observed. These results show that in vitro growth and transformation induce expression of the VEGF-B gene, particularly the 186 isoform, in line with previous observations (6). No VEGF-B species were secreted from the VEGF-B-deficient transformed MEFs. It is notable that most, if not all, MEFs do not express VEGF-B in the developing embryo (4).

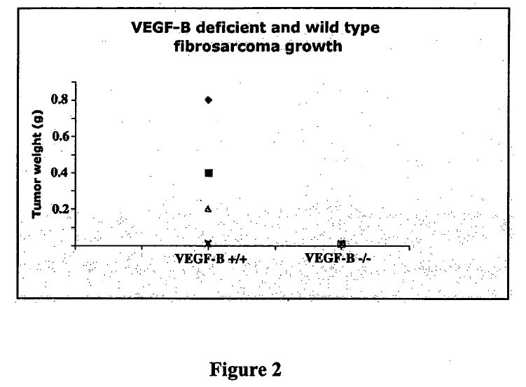

Injection of the two sets of transformed cells in syngeneic mice revealed that in the VEGF-B null group (VEGF-B −/− MEFs in VEGF-B −/− mice), the MEF cells did not grow into tumors and remained in their latent status (0/5 mice, see FIG. 2). In the VEGF-B positive group (VEGF-B +/+ MEFs in VEGF-B +/+ mice), however, most of the implanted MEF cells developed into sizable tumors (3/5 mice).

These data show that in vitro growth and transformation of MEFs lead to induction of VEGF-B expression, mainly of the 186 isoform. Furthermore, the VEGF-B expressing transformed MEFs generated large tumors in normal mice. On the contrary, the VEGF-B deficient transformed MEFs failed to generate any tumor at all in VEGF-B deficient animals. This shows that VEGF-B is required to initiate and support tumor growth, and is thus a suitable target for antagonistic therapy including but not limited to antibody-mediated therapy.

### Example 2

### Tumor Growth is Normal in VEGF-B-Deficient Mice and Up-Regulation of VEGF-B Stimulates Tumor Growth

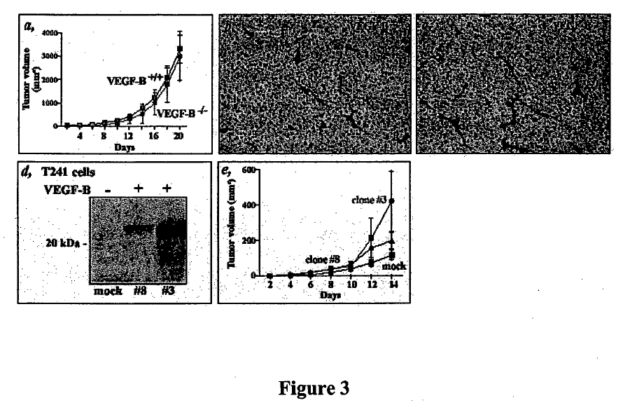

Murine T241 fibrosarcoma cells (which naturally express both the 167 and 186 isoforms of VEGF-B) were transplanted (1×10⁶ cells) into both wild-type (+/+) and VEGF-B KO mice (−/−) and the growth of the tumor was followed. As shown in FIG. 3(a), there was no difference is tumor growth, indicating that the host VEGF-B does not affect tumor growth.

An antibody to PECAM was used to stain tissue sections from the transplanted tumors. As shown in FIGS. 3(b) and (c) there was no difference in vessel density in cells transplanted into either WT (for VEGF-B) or VEGF-B KO mice.

Murine VEGF-B167 was cloned into the pcDNA3.1 expression vector which was transfected into T231 cells using lipofectin. Clones were selected using neomycin (400 micrograms/ml). FIG. 3(d) shows a western blot using an antibody to VEGF-B (which recognizes both 186 and 167 isoforms (see Aase et al., 1999, Developmental Dynamics 215:12-25). Lanes: mock-transfected (vector alone); and two different transfectant clones (#3 and #8) which have different levels of expression of VEGF-B167. The lane marked as #8 has intermediate levels of expression, while the lane marked as #3 has high levels of expression. VEGF-B₁₈₆ levels of expression are low.

Transfected cells in (d) were put into wild-type (WT) VEGF-B mice and the growth of the tumor was assessed. The results are shown in FIG. 3(e): Clone #3 showed accelerated growth of tumor versus mock transfected cells showing that tumor growth is dependent on VEGF-B₁₆₇ expression. The same experiment was done with the VEGF-B₁₈₆ isoform and preliminary results showed that tumor growth was even more accelerated.

### Example 3

### Monoclonal Antibodies Against VEGF-B Have Antagonistic Effects and Prevent Binding of VEGF-B to VEGFR-1

A. The Specificity of the Anti-VEGF-B Monoclonal Antibody MAB3372

Microtiter plates (Nunc Maxisorp) were coated with 1.0 μg/ml of recombinant VEGF-B protein (from Amrad Corp., Melbourne, Australia) in 50 μl 100 mM NaHCO₃ for overnight at 4° C. Residual binding capacity of the plates were blocked by incubating with PBS, supplemented with 3% BSA, for 0.5 hr at room temperature. The monoclonal anti-VEGF-B antibody MAB3372 (R&D Systems, Minneapolis) was diluted in 1% BSA in PBS at various concentrations, ranging from 0.03-1.0 μg/ml, and allowed to bind for 2 hr. Bound antibodies were incubated with 50 μl anti-mouse-IgG, conjugated with alkaline phosphatase (Sigma-Aldrich, 1:3000) for 2 hr. For visualization of the enzyme-conjugated secondary antibodies, 75 μl of NPP substrate (Sigma-Aldrich) was added. After stopping the reaction with 25 μl 0.5 M NaOH, the absorbance was measured at 405 and 650 nm with an ELISA plate reader.

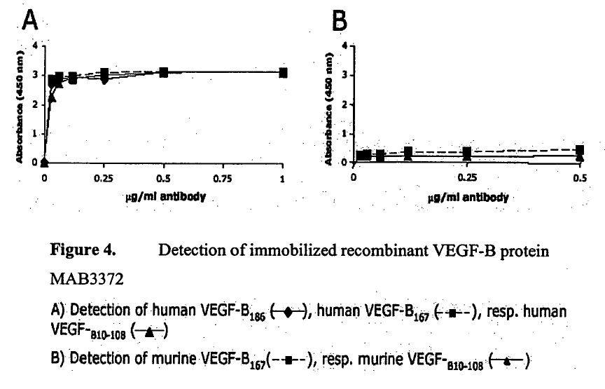

The results are shown in FIG. 4, which shows that MAB3372 recognizes both naturally occurring isoforms of VEGF-B, and that it also recognizes the truncated VEGF-B 10-108 protein, corresponding to the VEGF/PDGF growth factor domain only. However, this monoclonal antibody does not react strongly with mouse VEGF-B proteins.

B. VEGFR-1 Bioassay

Ba/F3 cells expressing the VEGFR-1/EpoR chimera (Makinen T, et al. Nat. Med. 2001, 7:199-205) were seeded in 96-well microtiter plates at 20,000 cells/well in quadruplicates supplied with 50 ng/ml of recombinant VEGF-B protein and with indicated concentrations of either anti-VEGF-B monoclonal antibody MAB3372 or sFlt-1 (R&D Systems). After 48 h, the viability of the cells was determined by adding tetrazolium salt WST-1 (4-[3-(4-Iodophenyl)-2-(4-nitrophenyl)-2H-5-tetrazolio]-1,3-benzene disulfonate; Roche Applied Science), followed by 2 hr of incubation at 37° C. Measurement of the absorbance was at 450 nm.

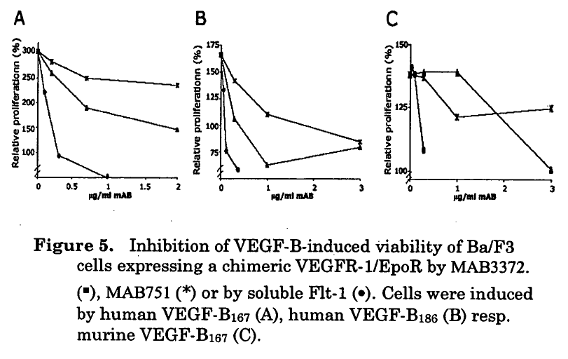

The results are shown in FIG. 5 and indicate that MAB3372 efficiently neutralized the effects of added human VEGF-B isoforms (FIG. 5A, B). MAB3372 was less efficient for mouse VEGF-B (FIG. 5C). Soluble Flt-1 (also known as soluble VEGFR-1) very efficiently neutralized both human and mouse VEGF-B. Anti-VEGF-B MAB751 (R&D Systems) partially neutralized the effect of human VEGF-B. Unrelated mouse Ig had no inhibitory effect (data not shown). These data show that MAB3372 is an efficient antagonist to VEGF-B, and that humanized derivatives of this monoclonal antibody may be used to antagonize the effect of VEGF-B in human disease conditions characterized by over-activity of VEGF-B.

C. VEGF-B₁₈₆ Overexpression Most Significantly Increases Tumor Growth

It is well established that recruitment of host-derived macrophages is a common feature in many cancers. Although the mechanisms underlying this phenomena are not understood, it is believed that general inflammatory reactions in tumors is of major importance in attracting macrophages into tumors. Macrophages express a variety of cytokines, growth factors, and proteases, and tumor-associated macrophages may contribute to the stromal reaction in tumors by releasing these factors, and thus promote tumor growth. Macrophages are known to express VEGFR-1, the receptor for VEGF-B. Therefore, it is important to determine whether expression of VEGF-B in tumors would affect recruitment of macrophages into tumors.

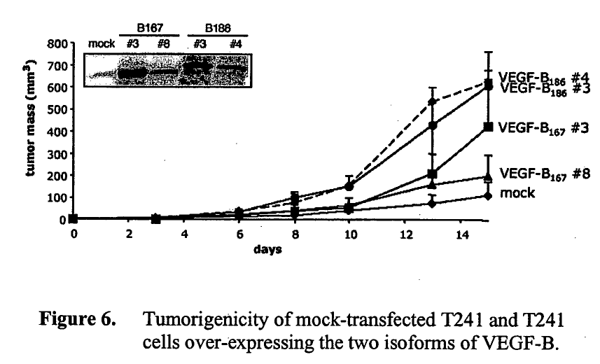

Tumors were established by growing T241 fibrosarcoma cells, modified to separately over-express the two isoforms of human VEGF-B, in C57/B1 mice. For each experiment 4 female 6-8-week-old mice were used. The expression level of exogenous VEGF-B in the various clones is shown in the Insert of FIG. 6. A suspension of 1×10⁶ tumor cells in 200 μl PBS (mock-transfected T241 fibrosarcoma cells, or T241 cells over-expressing VEGF-B₁₆₇ (cell clone #3 and #8), or VEGF-B₁₈₆ (cell clone #3 and #4)) were injected subcutaneously into the back of syngenic C57/B1 mice, and the growth rates of the resulting tumors were monitored for 15 days. For each experiment 4 female 6-8 week-old mice were used. The expression level of exogenous VEGF-B in the various clones was determined by immunoblotting. Conditioned media from mock-transfected, or VEGF-B over-expressing cell clones, were TCA-precipitated, and subjected to SDS-PAGE under reducing conditions, and immunoblotted using a affinity-purified rabbit Ig to VEGF-B.

The result are summarized in FIG. 6 and showed that T241 cells over-expressing VEGF-B₁₈₆ isoform generated faster growing tumors that did cells over-expressing VEGF-B₁₆₇, or mock-transfected cells.

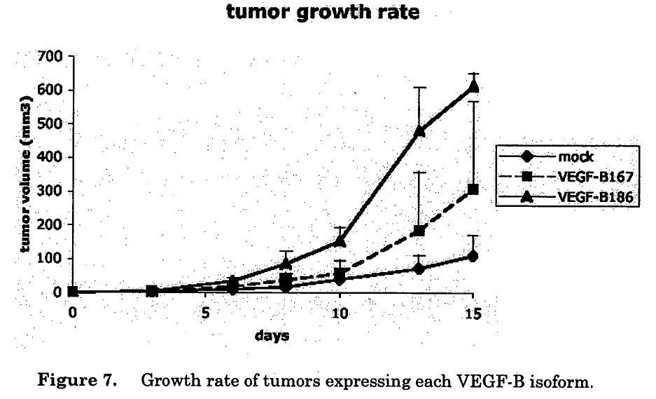

The data in FIG. 6 were pooled to show the growth rate of tumors in syngeneic mice expressing each isoform and that result, as shown in FIG. 7, shows that T241 tumors expressing VEGF-B₁₈₆ grow faster than those that express VEGF-B₁₆₇, which in turn grow faster than those that were mock-trasnfected.

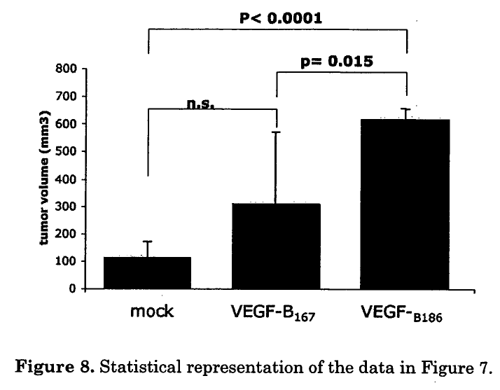

These data were further analyzed statistically and the results are shown in FIG. 8. Statistical representation of the data in FIG. 4 showing that VEGF-B₁₈₆ overexpressing tumors grow significantly faster than VEGF-B₁₆₇ overexpressing tumors and mock expressing tumors. No significant difference was seen between those overexpressing VEGF-B₁₆₇ and mock transfected T241 cells.

D. VEGF-B₁₈₆ Overexpression in Tumors Affects the Recruitment of Tumor-Associated Macrophages

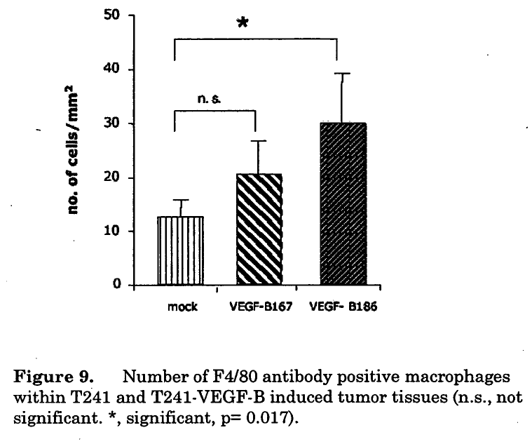

To mechanistically understand the effects of VEGF-B₁₈₆ overexpression in tumors the stromal reaction in the VEGF-B expressing tumors was studied. While the vessel density in the different tumors did not show any significant differences (using stainings with the endothelial cell marker PECAM, unpublished observations), it was found that invasion of macrophages, visualized using the marker F4/80, was dependent on the expression of the VEGF-B isoform. To visualize the F4/80 positive cells in the tumor tissues, tissue sections were prepared from fixed and paraffin-embedded tumor tissues using standard protocols. The antibody to the F4/80 antigen was obtained from Serotec, Oxford, United Kingdom. The stained cells were counted at random in 15 fields for one tumor section. Three tumors per clone and three sections per tumor were used for the analysis. The results are shown in FIG. 9, demonstrating that overexpression of VEGF-B isoforms, and in particular VEGF-B₁₈₆ isoform, stimulates the recruitment of macrophages into T241 tumors.

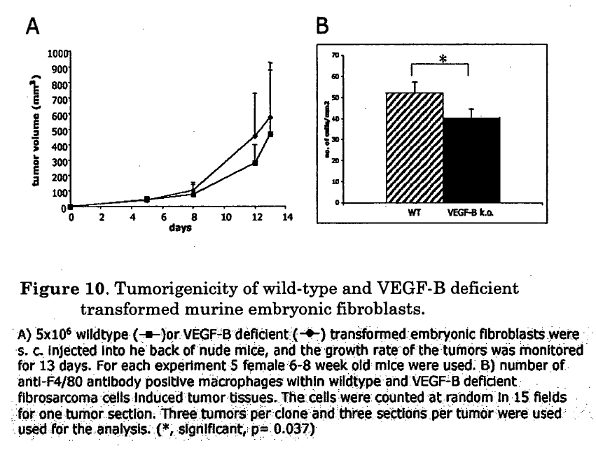

The ability of a VEGF-B deficient tumor to recruite macrophages was further explored. Transformed and wild-type mouse embryonic fibroblasts (MEFs, its generation is described supra) were injected into nude mice (5×10⁶ cells per mouse). The wild-type transformed MEFs grow slightly faster that did the VEGF-B-deficient MEFs in the nude mice (FIG. 10A). The resulting tumors were collected after 13 days, fixed in 4% paraformaldehyde in PBS, sectioned, and stained as above for the presence of macrophages using the antibodies to the F4/80 antigen. For each experiment 5 female 6-8 week old mice were used. FIG. 10B shows the number of F4/80 antibody positive macrophages within wild type and VEGF-B deficient fibrosarcoma tumor tissue. The cells were counted at random in 15 fields for one tumor section. Three tumors per clone and three sections per tumor were used used for the analysis. The results showed that tumors from VEGF-B deficient MEFs recruited less macrophages.

In summary, the above data show that VEGF-B over-expression, in particular VEGF-B₁₈₆, in tumor cells, accelerates the growth of tumors in animals. Thus VEGF-B is a rate limiting factor in tumor growth. VEGF-B expression in tumors partially regulates the recruitment of macrophages into the tumor. Macrophages are known to express growth factors and other factors that promote tumor growth. In addition, VEGFR-1 ligands are known to prevent the maturation of hematopoetic progenitor cells into dendritic, antigen presenting cells (Dikov et al. J. Immunol. 2005, 174:215-222, and Gabrilovich et al. Nat. Med. 1996, 2:1096-1103). Thus, lowering the amounts of VEGF-B in tumors may further allow the body to generate a stronger immune response to tumor antigens.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All references cited hereinabove and/or listed below are hereby expressly incorporated by reference.

### REFERENCES

1. Aase, K., von Euler, G., Li, X., Pontén, A., Thorén, P., Cao, R., Cao, Y., Olofsson, B., Gebre-Mehin, S., Pekny, M., Alitalo, K., Betsholtz, C., and Eriksson, U. Vascular endothelial growth factor-B-deficient mice display an atrial conduction defect (2001) Circulation 104, 358-364
2. Lodge, P. A., Haisch, C. E. & Thomas, F. T. A simple method of vascular endothelial cell isolation. Transplant Proc 24, 2816-7 (1992).
3. Hanahan, D., Lane, D., Lipsich, L., Wigler, M. & Botchan, M. Characteristics of an SV40-plasmid recombinant and its movement into and out of the genome of a murine cell. Cell 21, 127-39 (1980).
4. Aase, K., Lymboussaki, A., Kaipainen, A., Olofsson, B., Alitalo, K., and Eriksson, U. Localization of VEGF-B in the mouse embryo suggests a paracrine role of the growth factor in the developing vasculature (1999) Developmental Dynamics 215, 12-25
5. Olofsson, B., Pajusola, K., von Euler, G., Chilov, D., Alitalo, K., and Eriksson, U. Genomic organization of the mouse and human genes for vascular endothelial growth factor B (VEGF-B) and characterization of a second splice isoform (1996) The Journal of Biological Chemistry 271(32), 19310-19317
6. Li, X., Aase, K., Li, H., von Euler, G., and Eriksson, U. Isoform-specific expression of VEGF-B in normal tissues and tumors (2001) Growth Factors 19, 49-59
7. Aase, K., Lymboussaki, A, Kaipainen, A,. Olofsson, B., Alitalo, K. And Eriksson, U. Localization of VEGF-B in the Mouse Embryo Suggests a Paracrine Role of the Growth Factor in the Developing Vasculature (1999) Developmental Dynamics 215:12-25.

1. A method for inhibiting tumor cell formation or tumor cell growth, the method comprising administering to a patient in need thereof a pharmaceutically effective amount of an antagonist to VEGF-B.

2. A method according to claim 1, wherein said antagonist is specific to VEGF-B₁₈₆.

3. A method according to claim 1, wherein said antagonist is specific to VEGF-B₁₆₇.

4. A method according to claim 1, wherein said antagonist is anti-VEGF-B antibody.

5. The method according to claim 4, wherein the antibody is a monoclonal antibody.

6. The method according to claim 5, wherein the monoclonal antibody is humanized or chimeric antibody derived from MAB3372 or MAB751.

7. A method according to claim 4, wherein the antagonist comprises an isoform-specific antibody to VEGF-B₁₈₆.

8. A method according to claim 4, wherein the antagonist comprises an isoform-specific antibody to VEGF-B₁₆₇.

9. A method according to claim 4, wherein the antibody is a monoclonal antibody, or a Fab fragment, F(ab)₂ fragment, Fab′ fragment, F(ab′)₂ fragment, Fd fragment, Fd′ fragment or Fv fragments fragment thereof that specifically binds to VEGF-B.

10. A method according to claim 9, wherein the antibody is a humanized antibody.

11. A method according to claim 9, wherein the antibody is a human antibody.

12. A method according to claim 9, wherein the antibody is a domain antibody.

13. A method according to claim 9, wherein the antibody is a single-chain antibody.

14. A method according to claim 9, wherein the antibody is a chimeric antibody.

15. A method according to claim 1, wherein the VEGF-B antagonist comprises a small molecule antagonist.

16. A method according to claim 1, wherein the VEGF-B antagonist comprises an antisense nucleic acid molecule against a polynucleotide encoding VEGF-B, a ribozyme molecule specific against a polynucleotide encoding VEGF-B, an RNAi molecule specific against a polynucleotide encoding VEGF-B, or a nucleic acid molecule which forms a triple helix with a polynucleotide encoding VEGF-B.

17. A method according to claim 1, wherein the tumor cell is benign or malignant.

18. A method according to claim 1, wherein the patient is human.

19. A method according to claim 1, wherein at least one additional chemotherapeutic agent is administered to the patient.

20. A method according to claim 19, wherein the additional chemotherapeutic agent is administered together, before or after the administration of the antagonist to VEGF-B.

21. A method according to claim 19, wherein the additional chemotherapeutic agent comprises an antagonist against a member of the VEGF/PDGF family growth factors other than VEGF-B, and against VEGFR-1.

22. A method for inhibiting macrophage invasion of a tissue in a patient in need thereof, the method comprising administering to the patient a pharmaceutically effective amount of an antagonist to VEGF-B.

23. A method according to claim 22, wherein the method is for treating an inflammatory disease.

24. The method according to claim 23, wherein the inflammatory disease is selected from the group consisting of rheumatoid arthritis, inflammatory bowel disease, Crohn's disease, multiple sclerosis and psoriasis.

25. A pharmaceutical composition for treating tumor cell formation or tumor cell growth, comprising an antagonist to VEGF-B and a pharmaceutically acceptable excipient.
